(19) United States
(12) Patent Application Publication
(10) Pub. No.: US 2019/0224470 A1
(43) Pub. Date: Jul. 25, 2019

(54) CATHETER CLAMP

(71) Applicants: Tariq Chaudhry, Tampa, FL (US); Scott Hampton, Tarpon Springs, FL (US); Stephen Wilson Braun, Roswell, GA (US)

(72) Inventors: Tariq Chaudhry, Tampa, FL (US); Scott Hampton, Tarpon Springs, FL (US); Stephen Wilson Braun, Roswell, GA (US)

(21) Appl. No.: 15/779,303

(22) PCT Filed: Nov. 3, 2016
(86) PCT No.: PCT/US16/60355

§ 371 Date: May 25, 2018

Related U.S. Application Data
(60) Provisional application No. 62/250,159, filed on Nov. 3, 2015.

(51) Int. Cl. A61M 39/28

(52) U.S. Cl. CPC A61M 39/28; A61M 25/0606

(57) ABSTRACT

Provided are devices and methods for reducing or preventing fluid flow through a catheter lumen. For example, the devices and methods can be used to reduce blood flow through a catheter. Optionally, the catheter is an intravenous catheter.

### CROSS REFERENCE TO RELATED APPLICATIONS

This application is a non-provisional application that claims priority to U.S. Provisional Application No. 62/250,159 filed Nov. 3, 2015 which application is incorporated herein by reference.

### FIELD OF INVENTION

The present application relates to catheters and to devices and methods for reducing or preventing fluid flow through a catheter.

### BACKGROUND OF THE INVENTION

Hospital acquired infections can result from inadvertent spread of contaminants by hospital staff taking care of patients. Such infections lead to unanticipated prolonged hospital stays and increase healthcare costs in the form of expensive treatments.

Intravenous catheter placement carries a significant exposure risk of patient's blood to the hospital staff during the placement of an intravenous catheter, it is common for the patient's blood to leak out through the catheter end after a vein has been accessed and before the IV line is connected to the catheter. In addition to contaminating the patient's skin and the nurse's gloves, this creates an unpleasant sight for the patient. The nurse then connects the IV tubing and adjusts the flow-dial, thus inadvertently contaminating additional surfaces. During the hospital stay of a typical patient, multiple adjustments are made to the same intravenous line by additional hospital staff exposing even more personnel and surfaces to the patient's blood.

### SUMMARY OF INVENTION

Provided are devices and methods for reducing or preventing fluid flow through a catheter lumen.

An example device comprises a first clamping surface and a second clamping surface opposed to the first clamping surface. The surfaces at least partially define a substantially V-shaped gap to accept a portion of the catheter through which fluid flow is to be reduced or prevented. The first and second surfaces are moveable relative to one another to partially compress and occlude the catheter lumen to reduce or prevent fluid flow through the catheter.

Another illustrative device for reducing or preventing fluid flow through an intravenous catheter lumen includes first and second arcuate arms depending from a resilient biasing region which provides an outwardly biasing force on the first and second arms. A first clamping body extends from the first arcuate arm and has a first clamping surface and first engagement ramp thereon extending in a first direction. A second clamping body extends from the second arcuate arm and has a second clamping surface and a second engagement ramp thereon extending in a second direction opposite of the first clamping body.

In this embodiment the first and second engagement ramps are opposite and spaced apart from each other when the clamp is in an open position. At least one of the first and second clamping surfaces are frictionally engaged with each other, thereby defining a closed substantially fixed area in which the catheter is held.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

For a fuller understanding of the invention, reference should be made to the following detailed description, taken in connection with the accompanying drawings, in which:

FIGS. 1A and 1B are schematic illustrations of an intravenous catheter assembly;

FIGS. 2 through 4 are schematic illustrations of an example catheter clamping device;

FIG. 5 is a schematic illustration of an example catheter clamping device demonstrating that the length of the clamping surfaces is at least half the circumference the largest gauge of the catheter contemplated for use with the clamp;

FIG. 6 is a schematic illustration of an example catheter clamping device in a “fully pinched” position;

FIG. 7 is a schematic illustration of an example catheter clamping device in a “fully pinched” position;

FIG. 8 is a schematic illustration of an example catheter clamping device in a closed position demonstrating the off-set between the finger pads and the distal extent of the device (adjacent the contact point with the patient);

FIG. 9 is a schematic illustration of an example catheter clamping device in a “fully pinched” position demonstrating that the engagement protrusions are off-set at the end of the clamping device;

FIG. 10 is a schematic illustration of an example catheter clamping device in a closed position demonstrating that alignment surfaces provide for axially stability;

FIG. 11 is a schematic illustration of an example catheter clamping device in a closed position demonstrating that (1) the engagement protrusions prevent transverse and axial movement and (2) the engagement ramps are outward facing when the clamp is closed; and

FIG. 12 is a schematic illustration of an example catheter clamping device in a closed position demonstrating that the clamping surfaces are aligned when closed.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

As used throughout, by a “user” is meant an individual. Most commonly, the “user” will be clinician, technician or other health care provider. The term does not denote a particular age or sex.

Provided are devices and methods for reducing or preventing fluid flow through a catheter lumen. For example, the devices and methods can be used to reduce blood flow through a catheter. Optionally, the catheter is an intravenous catheter. The fluid is not limited to blood and the catheter is not limited to an intravenous catheter. The clamping devices described are optionally applied to any type catheter, or any fluid line, to reduce or eliminate fluid flow there through the catheter or fluid line's lumen.

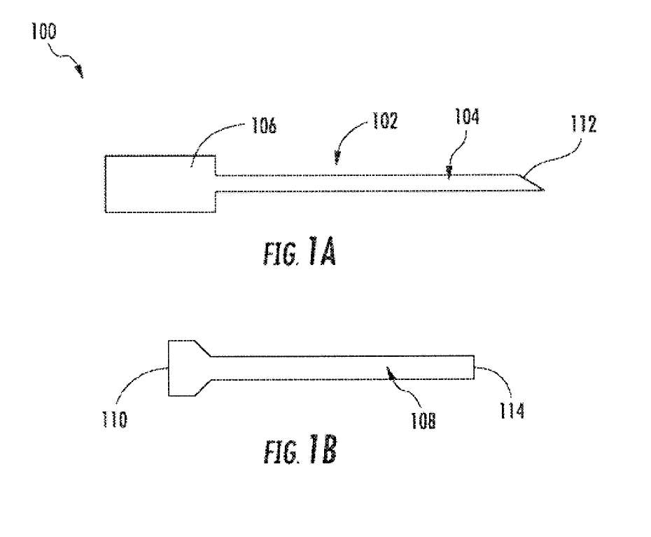

A typical intravenous catheter has two parts, a metal needle that punctures the vein and a plastic catheter which is left in the vein after the needle is pulled out. Illustrative devices can be found in U.S. Patent Publication No. US/20130304028 A1, which is incorporated herein by reference. Turning to the instant invention, FIGS. 1A and 1B are schematic illustrations of a catheter 108 and a needle 102 for use in accessing the vascular system of a subject.

The needle shaft 104 can be threaded through the catheter 108 lumen until the hub 106 contacts a proximal portion 110 of the catheter. In this arrangement, the needle shaft is positioned in the catheter 108 lumen and the sharp tip 112 of the needle 102 protrudes at least partially from the catheter lumen at a distal tip 114 of the catheter.

The sharp tip 112 can pierce the vessel wall to allow the tip 112 and at least a portion of the needle and catheter to enter the vessel lumen. Once at least a portion of the needle 102 and catheter 108 are located in the vessel lumen, the needle can be removed by sliding the needle distally from the catheter while the catheter remains in the vessel lumen. These steps are common to the placement of catheters in the vascular system to gain access to the intravascular space. The catheter may then be used to deliver therapeutics, such as fluids, to the subject.

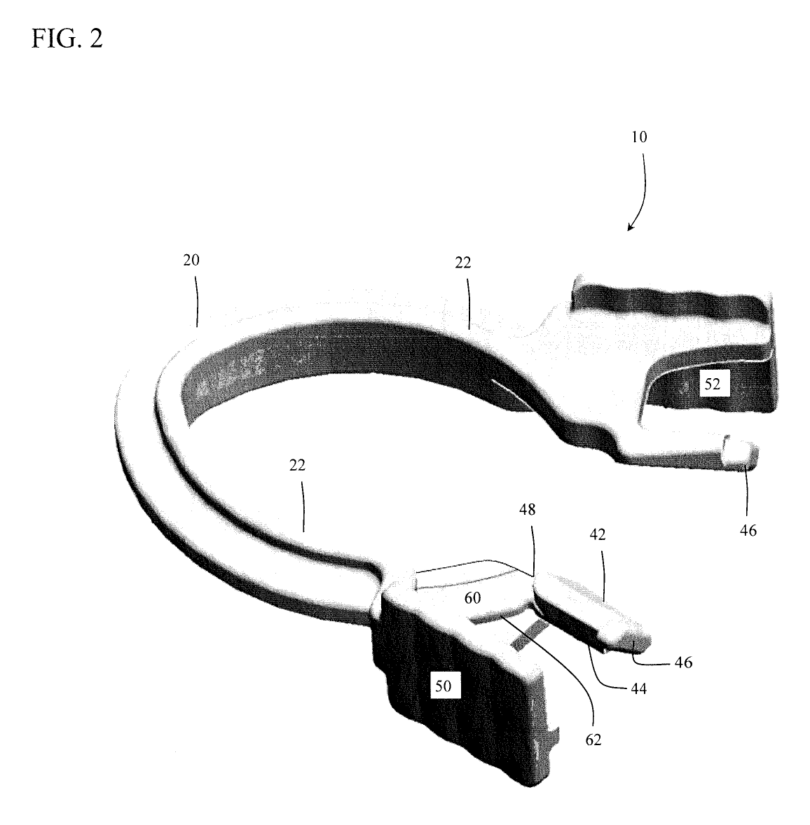

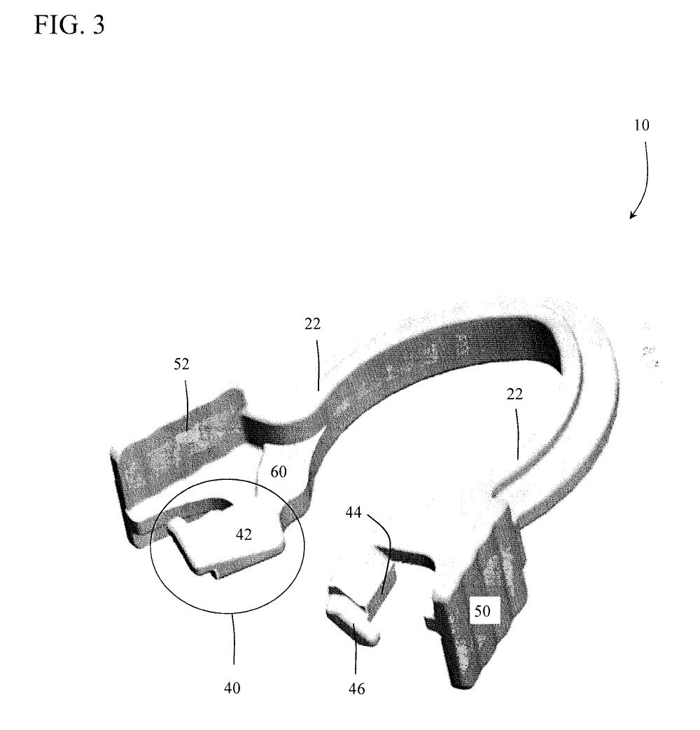

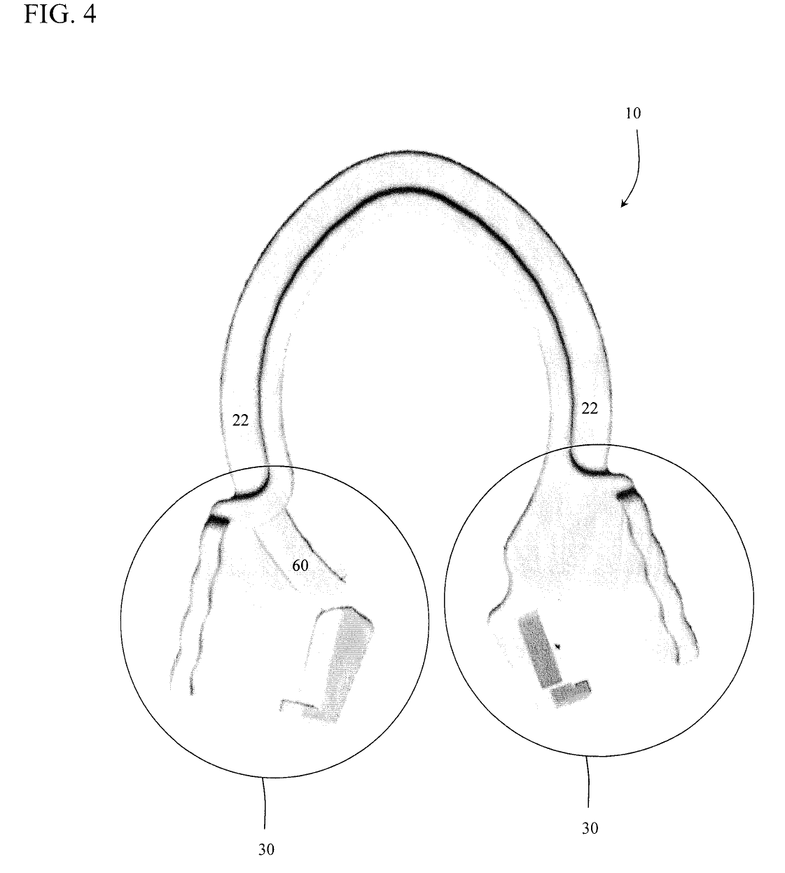

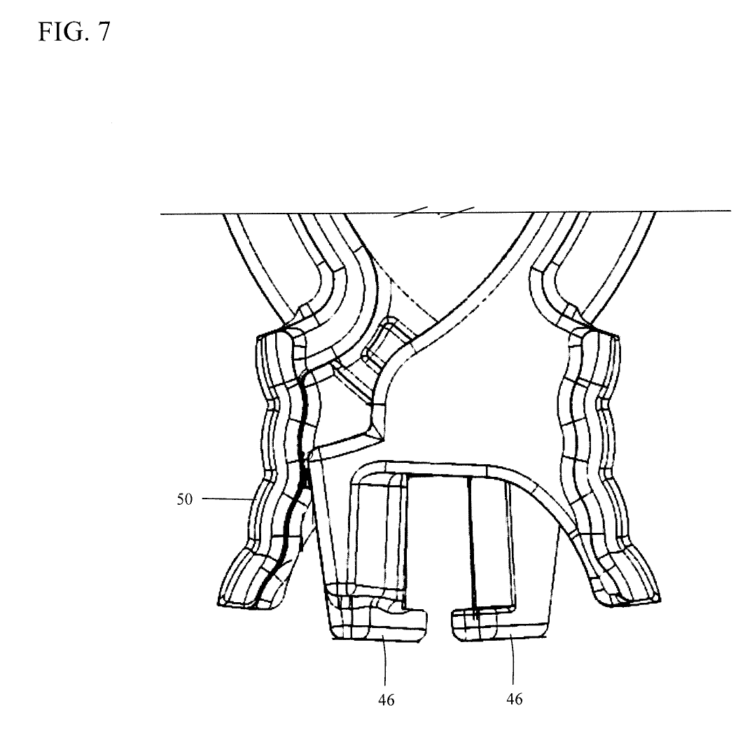

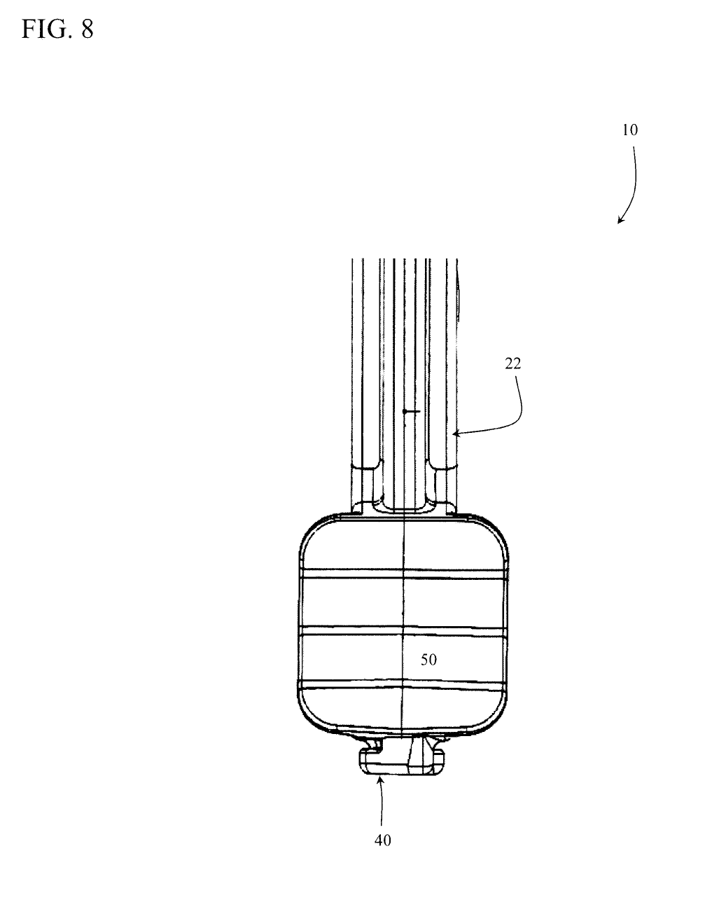

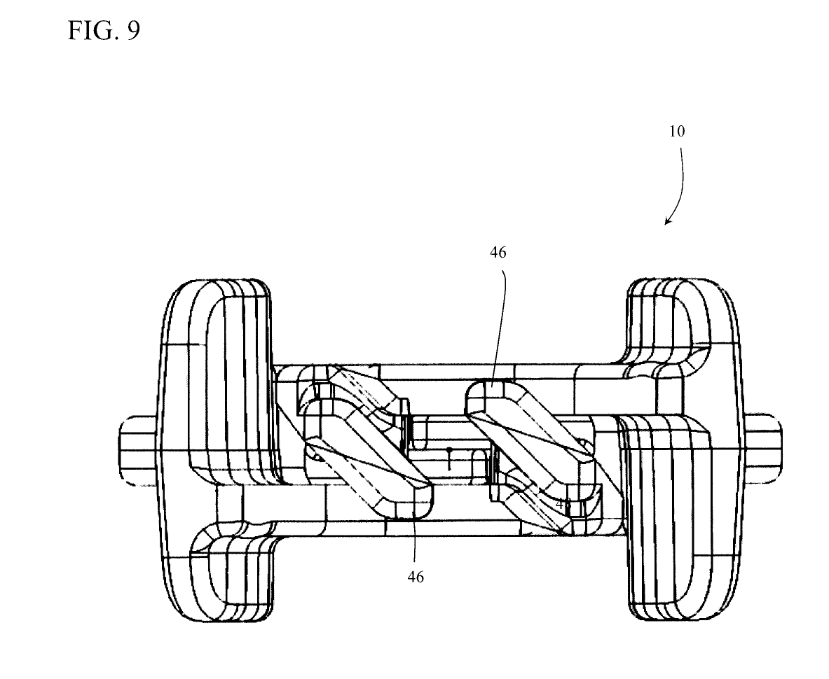

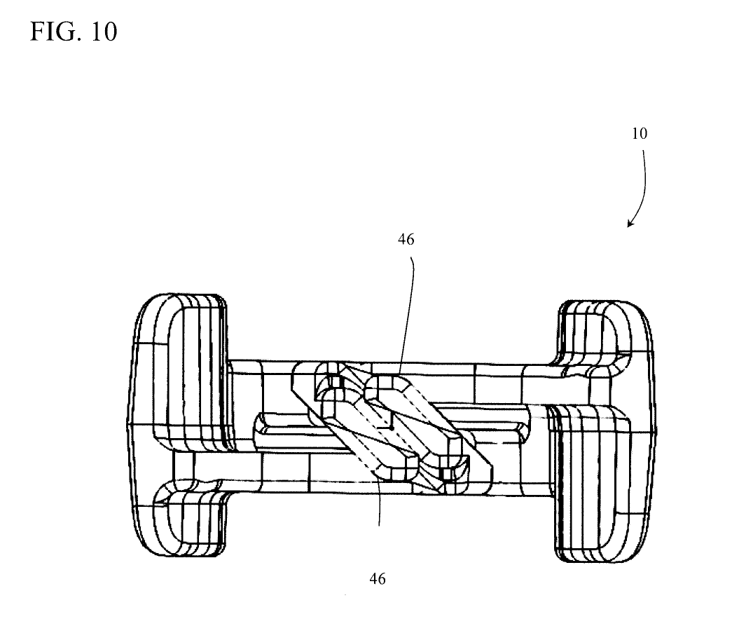

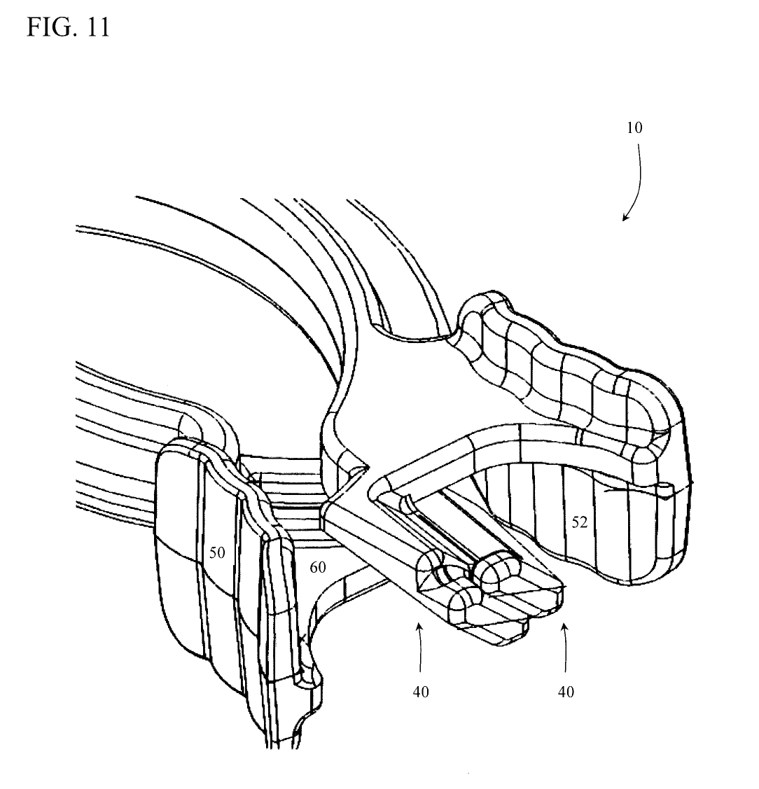

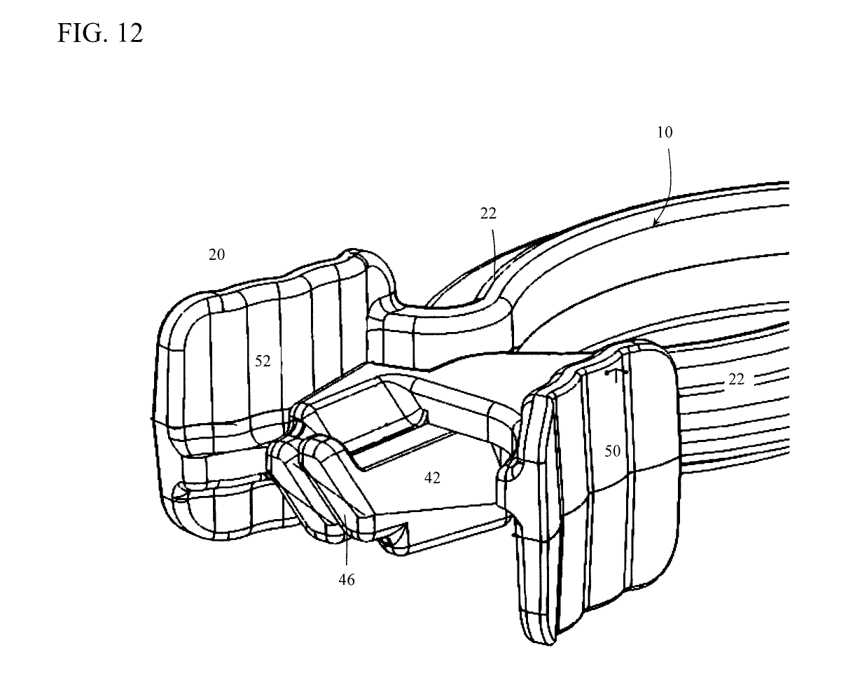

FIGS. 2 through 11 are schematic illustrations of an example clamping device. The clamping device is optionally positioned on a catheter when a needle is threaded through the lumen of the catheter. Upon removal of the needle, opposed clamping surfaces of the catheter clamp move relative to each other to narrow a space between the surfaces. Because a portion of the catheter without the needle is positioned in the space between the surfaces, and because the force applied to the catheter by the clamping surfaces is greater than the hoop strength of the catheter, once the needle is removed, the lumen of the catheter is narrowed or occluded by the crimping action of the clamping surfaces. This crimping action results in a reduction or elimination of blood flow through the catheter lumen when the catheter is operatively positioned in the vasculature of a patient.

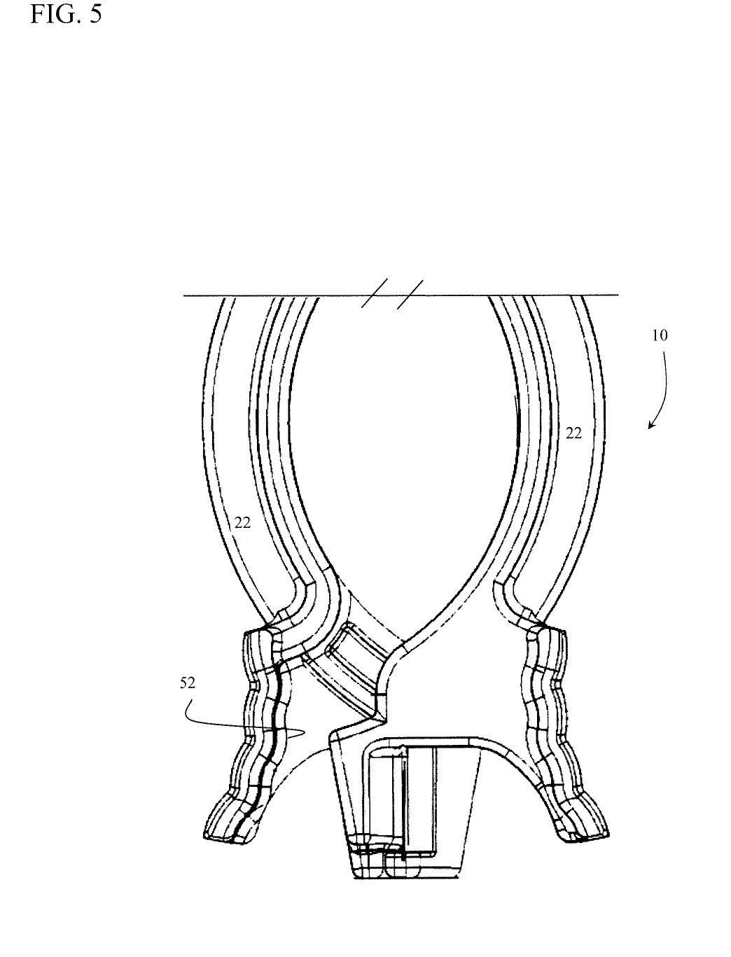

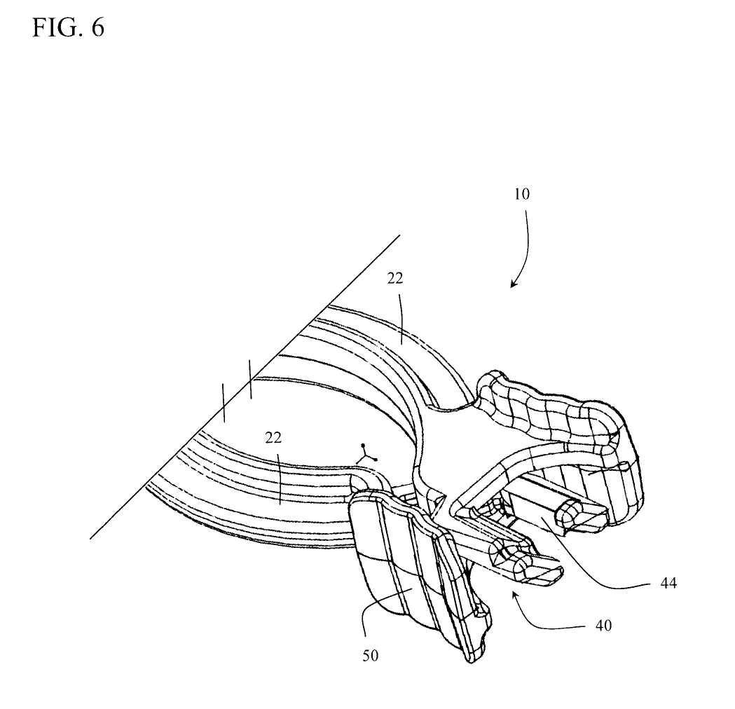

FIGS. 2 through 4 show an illustrative embodiment of clamping device 10 in an open configuration. FIGS. 5, 8 and 10 through 11 show an illustrative embodiment of clamping device 10 in a closed configuration. FIGS. 6, 7 and 9 show an illustrative embodiment of clamping device 10 in semi-open configuration.

Clamp 10 includes, generally, body 20 further including a pair of arms 22. Each arm 22 is attached to an opposing clamping body 30. Body 20 is preferably fabricated from a polymer having sufficient modulus of elasticity to allow clamp 10 to remain in the open position when no forces are acting upon it but such that it can be closed by normal hand pressure.

The elasticity of clamp 10 then applies sufficient outward pressure to keep the clamp in the closed position but not so much as to cause damage to a catheter within the clamp. The clamping bodies (30) are attached or are integral formed with biasing aims 22. This outward biasing force urges clamping members 30 to move in opposite directions relative to each other when positioned around the catheter.

In a preferred embodiment the modulus of elasticity is sufficient to apply two (2) to thirty (30) newtons between the clamping surfaces when the clamp is in the closed position. The application of force is a critical factor as insufficient force will not reduce the flow of fluids through the catheter, whereas excessive forces will cause damage to the catheter and interfere with the flow of fluids there through after the clamp is removed.

Each clamping body 30 further includes engagement body 40 and finger pad 50, which are connected alignment member 60. Finger pads 50 are provided to allow a user to pinch to overcome the modulus of elasticity of body 20. By pinching finger pads 50 with sufficient force to overcome the outwardly biasing force of body 20, the user can close the clamp by causing the clamping bodies 30 to move in the opposite direction as when moved by body 20. When clamping bodies 30 are moved apart a sufficient amount the catheter clamp is in a configuration that allows it to optionally be removed from the catheter, positioned on a catheter, or moved relative to the catheter.

The critical functionality of clamp 10, apart from the application of the predetermined force exerted by body 20, is achieved by the specific geometry of opposing and mating engagement bodies 40. As shown in FIG. 2B, engagement body 40 includes engagement ramp 42, clamping surface 44, protrusion 46 and alignment notch 48. In a preferred embodiment, the two opposing engagement bodies (40) are exactly symmetrical and opposite, but other embodiments are contemplated. In use, engagement ramps 42 are urged into contact by the user by applying finger force symmetrically against opposing finger pads 50 and overcoming the restorative force of the arcuate arms 22 of body 20.

As engagement ramps 42 come into contact, their respective geometries urge the engagement bodies (40), and by extensions all of clamping member 30, to undergo a slight displacement in opposite and generally axial directions until such time that each has travelled a sufficient distance that protrusion 46 on a first engagement body clears the corresponding protrusion 46 on the second engagement body. In a preferred embodiment, the angle of engagement ramps 42 is between about twenty (20) and seventy (70) degrees. As the finger force is continued and/or increased, the protrusions 46 are cleared. When force is applied to finger pads 50, over-travel of clamping members 30 is prevented when the leading edge of engagement ramp 42 contacts engagement stop 52 disposed on the opposite side of finger pad 50.

In a preferred embodiment, clamping surface(s) 44 have a narrow-tolerance construction which provides the necessary geometry to provide the necessary function. In this embodiment, the length of clamping surface(s) 44 is approximately half the circumference of the largest catheter to be used size plus up to about one (1) millimeter (mm). So for embodiments which contemplate the use of a clamp 10 in conjunction with fourteen (14) to twenty-four (24) gauge catheters, the length of clamping surface(s) 44 would be one (1) mm to five (5) mm.

The width of clamping surface(s) 44 should be at least 0.1 mm but can be as much as three (3) mm. Greater width requires a higher compression force but provides more stability when first applied. The width of clamping surface(s) 44 should not exceed five (5) mm, since widths in excess of five (5) mm could result in a gap between opposing clamping surfaces 44 that is too large to compress the catheter and prevent the movement of fluids there through.

Moreover, the shape and contour of clamping surface(s) 44 plays a critical role in the operation of clamp 10. Each clamping surface 44 should be a flat, rounded or convex (protruding) surface which is not sharp so that the catheter is not damaged. The edges of each clamping surface 44 may be chamfered, radiused, or curved as long as the resulting shape does not result in a sharp (crimping) edge that will contact the catheter.

When the user relaxes or ceases the applied force, the outwardly biasing force created by the modulus of elasticity of body 20 will cause the engagement bodies 40 to come into contact with the two clamping surfaces 44 in contact with the catheter held there between.

The alignment of clamping bodies 40, when in the closed position, is achieved by the synergistic construction of the finger pads 50 and alignment members 60. For example, axial alignment of engagement bodies 40 is achieved by alignment members 60. Each alignment member 60 includes a substantially planar surface having a raised wall 62. The height of wall 62 helps control axial alignment as the opposing alignment members 60 are in contact when the clamp is in the closed position

Alignment bodies 46 are respectively engaged by bodies 46 of the opposite engagement body 40, such that neither body is able to move axially when clamp 10 is in the closed position. Protrusions 46 overlap and nest such that clamping surfaces 44 are maintained in axial alignment and resist possible torques by providing a positive engagement of each member against alignment member 60 and protrusion 46 of opposite engagement body 40.

As discussed above; although the clamping surfaces 44 exert a force sufficient to hold the catheter in position relative to clamp 10, when the catheter is positioned there between—clamp 10 does not exert enough force on the catheter to affect the normal operation thereof when clamp 10 is not closed. For example, the force applied to the catheter by clamping surfaces 44 hold the catheter in place relative to the clamp and applies sufficient force to cause sufficient inward deformation (e.g. flattening) of the catheter sufficient to occlude the catheter's inner lumen.

When removing clamp 10 from a catheter, the user optionally pinches the device at finger pads 50 to separate clamping surfaces 44. Similarly, the first clamping body can be moved proximal relative to the second clamping body, or both clamping bodies can be moved with the second body being moved distally and the first being moved proximally. When clamping surfaces 44 are no longer in alignment for clamping, the pinching pressure may be released thereby allowing the device to expand to the open position.

Also provided are methods of reducing fluid flow, such as blood, through a catheter lumen. For example, a catheter clamping device is placed on an intravenous catheter before the catheter is inserted in a vein. Upon confirmation of the venous access, as the metal needle inside the catheter is pulled out, the spring loaded clamp shuts the plastic catheter and thus preventing leakage of the patient's blood, thus eliminating its exposure. Upon connection to the IV line, the clamp is taken off of the catheter.

Thus, the methods comprise placing a catheter with a needle positioned in its lumen into a vessel or other fluid containing space in a subject. A catheter clamp is positioned over the catheter such that at least a portion of the catheter is located in a slot defined by two opposed surfaces of the device that are moveable relative to one another. The needle is removed from the catheter to permit access to the vessel or fluid containing space and upon removal, the surfaces of the clamp move relative to one another to prevent or reduce fluid flow through the catheter.

An IV line or other therapeutic device or unit is positioned in communication with the catheter and once so positioned, the clamp is removed to allow fluid communication between the intravascular space or fluid containing space and the IV line or therapeutic device. The methods thereby reduce or prevent blood or fluid spillage from the catheter while establishing access to fluid filled spaces in the subject, such as the intravascular space.

The devices and methods described herein can be manufactured inexpensively and provide a barrier to the blood leaking through an intravenous catheter. In addition to the intravenous catheters, this device and methods can be used during the placement of the intra-arterial catheters as well as any other catheters placed for medical or surgical reasons.

The devices and methods do not require a change of the IV placement technique. Moreover, the devices and methods can be used for a wide variety of catheter sizes ranging from 24 gauge all the way up to the 14 gauge. The force that the opposed surfaces exert on the catheter can be varied depending on factors including the catheter size and the desired reduction of blood flow (e.g. from minimal reduction to prevention).

A wide variety of materials can be used to construct the device. For example, medical grade plastic can be used for single use, disposable clamps, whereas materials such as steel or titanium can be used if a reusable clamp is desired after sterilization.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

1. A device for reducing or preventing fluid flow through an intravenous catheter lumen, comprising:
a curved resilient biasing member forming an open ended structure, having a first and second ends, which provides an outwardly biasing force on the first and second ends;
a first clamping body extending from the first side of the biasing member and, having a first clamping surface and back surface thereon extending in a first direction; and
a second clamping body extending from the second side of the biasing member and having a second clamping surface and back surface thereon extending in a second direction opposite of the first clamping body;
wherein the back surface of the first clamping body is opposite and spaced apart from the back surface of the second clamping body when the clamp is in an open position;
wherein at least one of the first and second clamping surfaces is frictionally engaged thereby defining a slot area between the first and second ends of the biasing member to accept a portion of the catheter when the clamp is in a closed position; and
wherein the first and second clamping surfaces are moveable relative to one another to narrow the slot, and wherein the narrowed slot at least partially occludes the catheter lumen thereby reducing or preventing fluid flow through the catheter.

2. The device of claim 1, wherein the first clamping body extends downward from the first side of the biasing member and the second clamping body extends downward from the second end of the biasing member.

3. The device of claim 2, wherein the first and second clamping bodies having a width greater than the biasing member whereby each of the first and second clamping bodies are substantially flush with the biasing member on one side and the first clamping body extends from the first end of the biasing member in a first direction and the second clamping body extends from the second end of the biasing member in a second and opposite direction as the first clamping body.

4. The device of claim 2, wherein the first clamping surface is at least partially engaged with the second clamping surface and defining a closed substantially annular area between the first and second ends of the biasing member when the clamp is in a closed position.

5. The device of claim 1, wherein the first clamping body extends laterally from the first end of the biasing member and the second clamping body extends laterally from the second end of the biasing member.

6. The device of claim 5, wherein the first and second clamping bodies having a width substantially equal to the first and second ends of the biasing member.

7. The device of claim 5, where in the first clamping body extends from the first end of the biasing member in a first direction and the second clamping body extends from the second end of the biasing member in a second and opposite direction from the first clamping body.

8. The device of claim 4, wherein the first and second clamping bodies having a width greater than the first and second ends of the biasing member whereby each of the first and second clamping bodies are substantially flush with the first and second ends of the biasing member on one side and the first clamping body extends from the end of the biasing member in a first direction and the second clamping body extends from the second end of the biasing member in a second and opposite direction as the first clamping body.

9. The device of claim 4, wherein the first end of the biasing member has a depression, adjacent and spaced apart from the first clamping body, to retain a portion of the second clamping body and defining a space between the first and second clamping bodies when the clamp is in a closed position.

10. The device of claim 4, wherein the second end of the biasing member has a depression, adjacent and spaced apart from the second clamping body, to retain a portion of the first clamping body and defining a space between the first and second clamping bodies when the clamp is in a closed position.

11. The device of claim 1, wherein the clamping surfaces have lengths that are at least as long as the diameter of an intravenous catheter lumen.

12. The device of claim 1, wherein the tensile properties of the biasing member forcefully urges the first and second clamping bodies in compressional engagement relative to each other such that the clamping surfaces define a slot that is sized to hold an intravenous catheter with a needle in its lumen under force generated by the resilient properties of the biasing region, and such that a held intravenous catheter projects from the device such that it is accessible for insertion into the vasculature of a subject.

13. A device for reducing or preventing fluid flow through an intravenous catheter lumen, comprising:
a curved resilient biasing member forming an open ended structure, having a first and second ends, which provides an outwardly biasing force on the first and second ends;
a first clamping body extending from the first side of the biasing member and, having a first clamping surface and back surface thereon extending in a first direction; and
a second clamping body extending from the second side of the biasing member and having a second clamping surface and back surface thereon extending in a second direction opposite of the first clamping body;
wherein the back surface of the first clamping body is opposite and spaced apart from the back surface of the second clamping body when the clamp is in an open position;
wherein at least one of the first and second clamping surfaces is frictionally engaged thereby defining a slot area between the first and second ends of the biasing member to accept a portion of the catheter when the clamp is in a closed position; and
wherein the first and second clamping surfaces are moveable relative to one another to narrow the slot, and wherein the narrowed slot at least partially occludes the catheter lumen thereby reducing or preventing fluid flow through the catheter; and wherein the tensile properties of the biasing member forcefully urges the first and second clamping bodies in compressional engagement relative to each other such that the clamping surfaces define a slot that is sized to hold an intravenous catheter with a needle in its lumen under force generated by the resilient properties of the biasing region, and such that a held intravenous catheter projects from the device such that it is accessible for insertion into the vasculature of a subject.

14-23. (canceled)

24. A device for reducing or preventing fluid flow through an intravenous catheter lumen, comprising:
a curved resilient biasing member forming an open ended structure, having a first and second ends, which provides an outwardly biasing force on the first and second ends;
a first clamping body extending from the first side of the biasing member and, having a first clamping surface and back surface thereon extending in a first direction; and
a second clamping body extending from the second side of the biasing member and having a second clamping surface and back surface thereon extending in a second direction opposite of the first clamping body;
wherein the back surface of the first clamping body is opposite and spaced apart from the back surface of the second clamping body when the clamp is in an open position;
wherein at least one of the first and second clamping surfaces is frictionally engaged thereby defining a slot area between the first and second ends of the biasing member to accept a portion of the catheter when the clamp is in a closed position; and
wherein the first clamping body extends downward from the first side of the biasing member and the second clamping body extends downward from the second end of the biasing member.

25-34. (canceled)
